(19) United States
(12) Patent Application Publication
(10) Pub. No.: US 2021/0069403 A1
(43) Pub. Date: Mar. 11, 2021

(54) ONE PIECE ANASTOMOTIC CONNECTOR

(71) Applicant: PHRAXIS INC., St. Paul, MN (US)

(72) Inventor: Alexander S. YEVZLIN, Black Earth, WI (US)

(21) Appl. No.: 16/957,465

(22) PCT Filed: Feb. 8, 2019
(86) PCT No.: PCT/US19/17200

§ 371 Date: Jun. 24, 2020

Related U.S. Application Data
(60) Provisional application No. 62/628,032, filed on Feb. 8, 2018.

(51) Int. Cl. A61M 1/36

(52) U.S. Cl. CPC A61M 1/3655

(57) ABSTRACT

A one piece anastomotic connector is provided. The one piece anastomotic connector is substantially T-shaped in cross-section and has an arterial portion and a venous portion, the arterial portion is implantable into an arterial passageway and the venous portion implantable into a venous passageway. The venous portion and the arterial portion are integrally formed. The arterial portion allows for undisturbed, full flow of blood into extremities of a patient downstream the anastomotic connector.

### FIELD OF THE INVENTION

This invention relates to anastomotic connector devices. In particular, this invention relates to a vascular access device for use in hemodialysis and other procedures, such as in the cardiovascular field, where short-term and long-term access is required.

### BACKGROUND OF THE INVENTION

In the United States alone, approximately 400,000 people have end-stage renal disease requiring chronic hemodialysis. Hemodialysis replaces kidney function by removing toxins from the blood that are normally removed by healthy kidneys. In order to effectively remove toxins, blood must he passed at a high blood flow rate through a hemodialysis machine. This high blood flow is best achieved by the creation of a permanent vascular access site that includes an arteriovenous (AV) anastomosis in which a vein is attached to an artery to form a high-flow shunt or fistula.

Typically, a vein may be directly attached to an artery, but it takes from six to eight weeks before the fistula has sufficiently matured (time between placement and cannulation for dialysis) to provide adequate blood flow for use with hemodialysis. Moreover, a direct anastomosis may not be feasible in all patients due to anatomical considerations. Other patients may require the use of artificial graft material to provide an access site between the arterial and venous vascular systems. Because of the length of time required for a fistula to mature a patient needing dialysis Will typically require a temporary access device, such as a Quinton catheter, to be inserted for hemodialysis access until the fistula has matured. The use of a temporary catheter access exposes the patient to additional risk of bleeding and infection, as well as discomfort, and is associated with a 91% higher mortality rate compared to fistulas. In trying to increase the prevalence of fistulas in the U.S., a proportional rise in catheter use has been documented.

Therefore, what is needed is an improved vascular access device that addresses the foregoing problems.

### BRIEF SUMMARY OF THE INVENTION

The present invention addresses the foregoing problems by allowing a minimally invasive connection to be created between an artery and vein in the arm of a patient without the need for creation of a surgical anastomosis.

In one aspect of the invention a device for connecting an artery and vein to create an arteriovenous anastomosis for hemodialysis is provided. The connector device is a one piece anastomotic connector including a lumen therewithin and an arterial portion and a venous portion. The anastomotic connector device may comprise a stent constructed of a shape-memory material and optionally covered with a non-porous material to prevent leakage. The anastomotic connector is configured to preserve blood flow to the hand.

In another aspect of the invention, the arterial portion includes a first arterial end and a second arterial end. The arterial portion is sized and designed to fit into a fluid arterial passageway of a patient and most preferably the radial artery of a patient (˜3-5 mm diameter). Thus, the outer diameter of the arterial portion is slightly larger than the inner diameter of the radial artery such that when the arterial portion is expanded in the radial artery, the radial force of the arterial portion exerts a compressive force against the arterial wall so that it remains in place.

In another aspect of the invention, the venous portion is contiguous and integrally formed with the arterial portion to form the one piece connector. The venous portion is sized and designed to fit into a fluid venous passageway of a patient and most preferably the cephalic vein of a patient (5-7 mm diameter). The venous portion may include an anchoring device at the open end thereof. The anchoring device may have one or more barbs that are configured to secure the venous portion 18 within the fluid venous passageway. In such case, the optional non-porous material would not cover the anchoring device as can be appreciated by those of skill in the art. In the ease of a plurality of barbs comprising the anchoring device, the barbs may circumferentially surround the open end of the venous connector.

Depending on the anatomy of a particular patient the length of the one piece connector may be from approximately 4 cm to 10 cm.

In another aspect of the invention, the one piece connector is delivered as unit into the radial artery and cephalic vein through a 3 cm incision via the Seldinger technique over three separate guide wire/sheath systems.

In another aspect of the invention, the anastomotic connector couples the radial artery to the cephalic vein in the location of the forearm without the need for graft material connecting two separate pieces as with conventional devices.

In another aspect of the invention, a minimally invasive way to create a fistula using a one piece connector is provided thereby eliminating the need for graft material to couple two connectors together as with conventional anastomotic connectors.

In another aspect of the invention, the geometry of the one piece anastomotic connector allows continuous and uninterrupted arterial or venous flow during use for dialysis or other applications, thereby elimminating or substantially reducing any loss of circulation to the downstream, distal extremities in particular the hand. Stated alternatively, the geometry of the one piece anastomotic connector allows “full” or undisturbed flow into the hand of the patient, which is downstream the connector. Thus, distal arterial flow is not “cut-off” due to the presence of the one piece anastomotic connector.

Another advantage is that the anastomotic connectors of the invention may be implanted percutaneously rather than with an “open surgery” approach. The implantation method is therefore less invasive for the patient and faster for the surgeon.

Yet another advantage is that the one piece connector allows for maturation of the distal vein in preparation for secondary AVF while avoiding a central dialysis catheter.

### BRIEF DESCRIPTION OF THE DRAWINGS

For a better understanding of the invention, and to show how the same may be carried into effect, reference will now be made, by way of example, to the accompanying drawings, in which:

FIG. 1 an illustration of the one piece anastomotic connector in accordance with the invention implanted in artery and vein.

FIG. 2 is an illustration depicting the delivery of the one piece anastomotic connector into an artery and a vein.

### DETAILED DESCRIPTION OF THE INVENTION

Generally speaking, the present invention is directed to an anastomotic connector structured to connect an artery and a vein without the need for a graft material. The anastomotic connectors in accordance with the present invention may be placed using the Seldinger technique in the radial artery and cephalic vein, and may be fabricated from any biocompatible material suitable for implantation into the human body. A preferable material is so called shape-memory materials.

Numerous structural variations of an anastomotic connector device are contemplated and within the intended scope of the present invention. As those skilled in the art will appreciate, the teachings with regard to the disclosed embodiment may apply to the other embodiments even if not specifically stated.

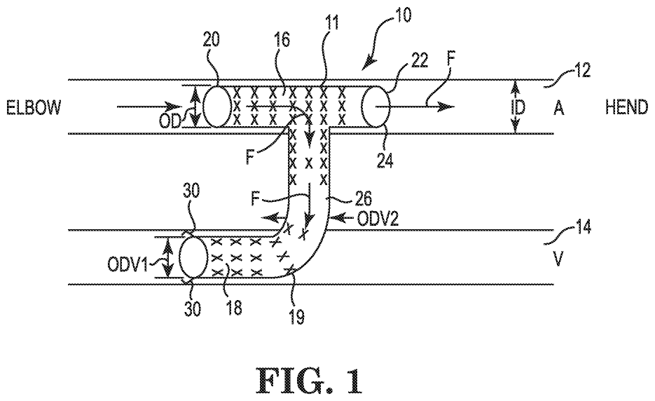

FIG. 1 is an illustration of the T-shaped anastomotic connector 10 in accordance with the invention. As illustrated in FIG. 1, anastomotic connector 10 generally includes a main body 11 with a side tubular portion 26 extending radially outward therefrom. Main body 11 includes an open first arterial end 20 and an open second arterial end 22. Side tubular portion 26 extends substantially perpendicular from main body 11 about midway between first arterial end 20 and second arterial end 22. For this reason, anastomotic connector 10 may be referred to as a “T” connector due to its “T” shaped structure. It can been seen that side tubular portion 26 is directly and integrally formed and in fluid communication with the main body 11 of the arterial portion 16 and with the main body 19 of the venous portion 18.

The anastomotic connector 10 comprises a one piece anastomotic connector that advantageously connects the radial artery 12 to the cephalic vein 14 in the location of the forearm without the need for graft material. The arterial portion 16 of one piece anastomotic connector 10 includes lumen 24 that advantageously allows uninterrupted blood flow F from the radial artery 12 to the hand and into the cephalic vein 18.

As noted, one piece anastomotic connector 10 includes an arterial portion 16 and a venous portion 18 integrally formed with the arterial portion 12. The arterial portion includes a first arterial end 20 and a second arterial end 22.

The arterial portion is sized such that the outer diameter OD of the main body 11 is slightly larger than the inner diameter ID of the radial artery. Thus, when the arterial portion expands into place it exerts a compressive force against the arterial wall to ensure it does not dislodge.

Venous portion 18 is integrally formed with arterial portion 16 via side tubular portion 26. Venous portion 18 may optionally include an anchoring device 30. Anchoring device 30 may comprise one or more hooks, barbs, tines and other types of curved or angled fasteners designed to anchor the venous portion 18 to an inner surface of the venous wall. The barbs may circumferentially surround the open end 28 of the venous portion 18 and be angled outwardly from a longitudinal axis. The barbs may penetrate the inner venous wall or simply lie adjacent the inner venous wall exerting a force thereon without penetration.

The anastomotic connector accordance With the vent on is substantially tubular shaped and includes a resilient side tubular portion 26. Those of skill in the art will appreciate that because the arterial portion e venous portion are integrally formed via side tubular portion the construct is of the same material. The arterial portion 16 is sized to fit within the radial artery and the venous portion 18 is sued to fit within the cephalic vein. The venous portion may have an outer diameter of from 5 to 7 mm while the arterial portion may have an outer diameter of from 3 to 5 mm.

The anastomotic connector 10 may be constructed of a flexible or resilient material. Shape memory alloys such as NITINOL, stainless steel, and various polymers may be used. Those of skill in the art will appreciate that NITINOL may be preferable due to its high yield strain. However, any suitable self-expanding material may be used as will be appreciated by those of ordinary skill in the art.

One class of materials which meets the foregoing qualifications is so-called shape memory alloys. Such alloys tend to have a temperature induced phase change which will cause the material to have a preferred configuration which can be fixed by heating the material above a certain transition temperature to induce a change in the phase of the material. When the alloy is cooled back down, the alloy “remember” the shape it was in during the heat treatment and will tend to assume that configuration unless constrained from so doing. One particularly preferred shape memory alloy for use in the present method is Nitinol, an approximately stoichiometric alloy of nickel and titanium, which may also include other minor amounts of other metals to achieve desired properties. NiTi alloys such as Nitinol, including appropriate compositions and handling requirements, are well known in the art and such alloys need not be discussed in detail here. Such NiTi alloys are preferred, at least in part, because they are commercially available, have a high yield strain and more is known about handling such alloys than other known shape memory alloys. NiTi alloys are also very elastic—they are said to be “superelastic” or “pseudoelastic.” This elasticity will help the anastomotic connector in accordance with the invention to return to a pre-set expanded configuration for deployment into a blood vessel. However, any suitable self-expanding material may be used as will be appreciated by those of ordinary skill in the art.

In forming the exemplary anastomotic connector 10 of the invention, an appropriately sized piece of the metal fabric may be cut from a larger piece of fabric which is formed, for example, by braiding wire strands to form a long tubular braid. The dimensions of the piece of fabric to be cut will depend, in large part, upon the size and shape of the connector to be formed therefrom. It is contemplated that the size and shape of the connector may vary depending on whether the use is for adults or children.

When cutting the fabric to the desired dimensions care should be taken to ensure that the fabric will not unravel. In the case of tubular braids formed of NiTi alloys, for example, the individual wire strands will tend to return to their heat-set configuration unless constrained. If the braid is heat treated to set the strands in the braided configuration, they will tend to remain in the braided form and only the ends will become frayed. However, it may be more economical to simply form the braid without heat treating the braid since the fabric will be heat treated again in forming the medical device. Alternatively, one can solder, braze, spot, laser treat or otherwise affix the ends of the desired length together (e.g. with a biocompatible cementitious organic material) before forming the connectors.

Once an appropriately sized piece of the tubular metal fabric is obtained, it may be inverted onto itself and deformed to generally conform to a surface of a molding element. As will be appreciated, so deforming the fabric will reorient the relative positions of the strands of the metal fabric from their initial order to a second, reoriented configuration. The shape of the molding element should be selected to deform the fabric into substantially the shape of the exemplary anastomotic connector.

The molding element can be a single piece, or it can be formed of a series of mold pieces which together define the surface to which the fabric will generally conform. The molding element can be positioned within a space enclosed by the fabric or can be external of such a space, or can even be both inside and outside such a space.

Those of skill in the art will, also appreciate that the construct of the anastomotic connector 10 may comprise a single layer of shape-memory material without departing from the scope of the invention.

The anastomotic connector 10 in accordance With the invention is configured to be optionally coated with a biocompatible material to prevent leakage of blood. The biocompatible material may be a woven material or other constructs known to those of skill in the art. Such biocompatible materials may include, but are not limited to, expanded Polytetrafluoroethylene (“ePTFE”), polyester, porcine vessel, THORALON (a self-sealing polyurethane material), Polytetrafluoroethylene (“PTFE”) modified with urea (such as the VECTRA. graft), silicone composites, or various other plastics and elastomers or combinations thereof. The anchoring device 30 positioned at the open end 28 of the venous portion 18 may be configured to remain free of coating to enhance its ability to engage the inner wall of the vein.

The biocompatible covering 32 of the anastomotic connector 10 may be formed on an inner surface of the frame-like structure of the anastomotic connector. In other embodiments biocompatible covering 32 may be formed on an outer surface of the frame-like structure, or alternatively on both the inner and outer surfaces of the mesh frame without departing from the intended scope of the present invention.

Additionally, it may be preferable to provide the anastomotic connector 10 of the invention with an inner surface that is contoured to allow smooth arterial or venous blood flow into and out of the connector. As those of ordinary skill in he art will appreciate, providing a non-thrombogenic surface minimizes the creation of recirculation or stagnation zones with high shear or dwell times that could otherwise lead to clotting.

It is also contemplated that the inner or outer surface of the anastomotic connector 10 be configured to deliver and release therapeutic substances such as anti-microbial agents, anti-inflammatory agents, anti-proliferative agents (e.g. taclipaxel), growth factors, stem cells, collagen and the like. Those of ordinary skill in the art will appreciate that these therapeutic agents may be coupled with the connector and/or the external or internal surface of the connector by means such as being encased or embedded in the optional biocompatible coating, applied to a textured external surface of the connector; contained within pockets of the connector on either an internal or external surface, and the like.

It is contemplated that the frame-like structure of the anastomotic connector may include almost any configuration of struts and connectors known to those of skill in the art. Some of those configurations are disclosed in U.S. Pat. Publn. No. 20170196676. The column pitch of the venous portion 18 of the anastomotic connector may be loose or substantially equivalent along the length of the venous portion and the tubular main body portion 11 to allow it to easily bend. The column pitch of the arterial portion may vary along the length of the arterial portion.

The arterial portion may include a plurality of piercing or non-piercing flanges 23 circumferentially disposed about the first arterial end 20 and the second arterial end 22. The flanges 23 may be integrally formed with tubular main body 11. The flanges 23 may be configured to bend and extend radially outward at an angle greater or less than 90 degrees away from the longitudinal axis of the arterial portion 16. The flanges 23 may be configured to spread from a first reduced configuration to a second expanded configuration to anchor the arterial portion 16 against the inner wall of an arterial fluid passageway. The struts and connectors that form the arterial portion 16 may include a variable cutting pattern along the length thereof.

The outer diameter ODVI of the open end 28 of venous portion 18 may be greater than the outer diameter OVD2 of body portion 26 to ensure it is property seated in an interference fit within a venous fluid passageway. In addition, the outer diameter OD of the entire arterial end 16 including first arterial end 20, second arterial end 22 and main body portion 11 is sized to ensure an interference fit within the arterial fluid passageway. The anastomotic connector in accordance with the invention may have a column pitch that is substantially equivalent along the length of the anastomotic connector or the anastomotic connector has a column pitch that varies along the length of the anastomotic connector.

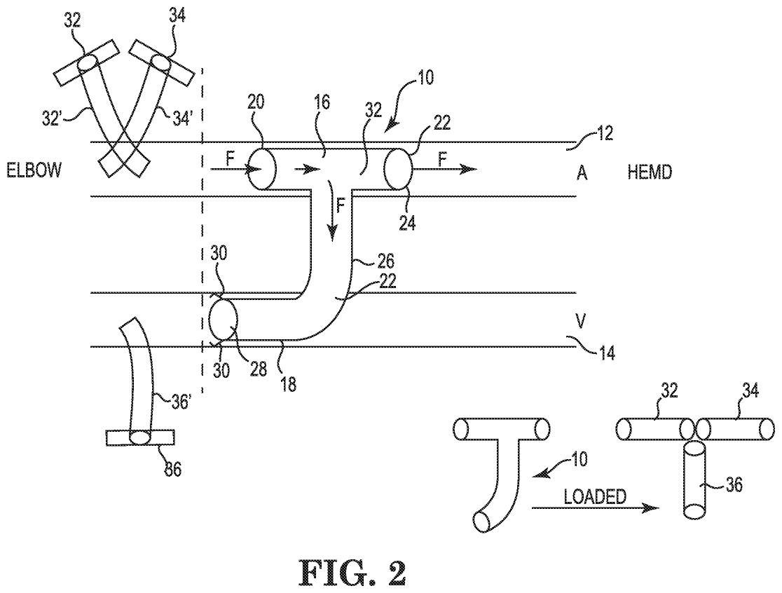

Referring now to FIG. 2, the one piece anastomotic connector is delivered as a unit into the radial artery and cephalic vein through a 3 cm incision via the Seldinger technique over three separate delivery component/sheath systems. The desired vessel or cavity is punctured with a sharp hollow needle, with ultrasound guidance if necessary. A round-tipped guidewire is then advanced through the lumen of the needle, and the needle is withdrawn. The anastomotic connector may then be passed into the vessel and the guidewire is withdrawn. Upon completion of the desired procedure, the sheath is withdrawn. In certain settings, a sealing device may be used to close the hole made by the procedure.

The delivery component/sheath systems comprise a soft material that can easily be peeled away and removed. Such materials may include polymers such as highly kink-resistant PTFE materials. The one piece anastomotic connector device 10 in accordance with the invention is first loaded into the three part delivery system 32, 34, 36 as shown in FIG. 2. The first delivery component 32 inserted into the first peel away delivery sheath 32′. The second delivery component 34 is inserted into the second peel away sheath 34′ and the third component 36 is loaded into the third delivery sheath 36′. The first and second sheathed components 32, 32′, 34, 34′ are positioned within the artery through an incision made therein. The third sheathed component 36, 36′ is positioned in the vein. The sheaths 32′, 34′, 36′ are peeled away. When the device 10 is properly positioned within the radial artery and cephalic vein the components 32, 34 and 36 are peeled away from the device 10.

The present invention provides a minimally invasive way to create a fistula using a one piece connector without the need for graft material to couple two separate arterial and venous pieces as with conventional devices.

Based upon the present disclosure and after viewing the exemplary embodiment of the anastomotic connector 10 presented herein, the many advantages and benefits provided by the invention will be appreciated by those of ordinary skill in the art. The geometry of the one piece anastomotic connector 10 allows continuous and uninterrupted arterial and venous flow during use for dialysis or other applications, thereby eliminating or substantially reducing any loss of circulation to the downstream, distal extremities in particular the hand. Stated alternatively, the geometry of the one piece anastomotic connector allows “full” flow into the hand of the patient, which is downstream the anastomotic connector. Thus, distal arterial flow is not “cut-off” due to the presence of the one piece anastomotic connector.

Another advantage is that the anastomotic connectors of the invention may be implanted with minimally invasive surgery rather than with an “open surgery” approach. The implantation method is therefore less invasive for the patient and faster for the surgeon.

Although the present invention has been described with reference to preferred embodiments, workers skilled in the art will recognize that changes may be made in form and detail without departing from the spirit and scope of the invention.

What is claimed:

1. An anastomotic connector comprising:
a one piece anastomotic connector having an arterial portion having a first open end and a second open end and implantable into an arterial passageway, a venous portion having a single open end implantable into a venous passageway, the arterial portion and venous portion coupled by an integrally formed tubular body portion, said one piece anastomotic connector being T-shaped in cross section,
wherein an outer diameter of the venous portion is greater than an outer diameter of the arterial portion and the first and second openings of the arterial portion allow for undisturbed, full flow of blood into extremities of a patient downstream the anastomotic connector.

2. The anastomotic connector of claim 1 wherein the tubular body portion extends radially outward from a mid-portion of the arterial portion.

3. The anastomotic connector of claim 1 wherein said venous portion includes an anchoring device on the open end that extends radially outwardly at an acute angle from a longitudinal axis of the venous connector.

4. The anastomotic connector of claim 3 wherein said anchoring device is configured to lie adjacent a venous vessel wall.

5. The anastomotic connector of claim 3 wherein said anchoring device is configured to penetrate a venous vessel wall to seat said venous portion in a venous passageway.

6. The anastomotic connector of claim 1 wherein said anastomotic connector is coated with a fluid impermeable material.

7. The anastomotic connector of claim 1 wherein said anastomotic connector is formed from a shape memory material.

8. The anastomotic connector of claim 5 wherein said fluid impermeable material is woven.

9. The anastomotic connector of claim 5 wherein said fluid impermeable material is a polymeric material.

10. The anastomotic connector of claim 5 wherein said fluid inmpermeable material is deposited onto said anastomotic connector by electrospinning.

11. The anastomotic connector of claim 5 wherein said fluid impermeable material is deposited onto said device by extrusion.

12. The anastomotic connector of claim 5 wherein said fluid impermeable material covers the entirety of said anastomotic connector.

13. The anastomotic connector of claim 5 wherein said fluid impermeable material covers the arterial portion, the tubular body portion and the venous portion excluding the anchoring device, which remains uncoated.

14. The anastomotic connector of claim 1 wherein the anastomotic connector has a column pitch that is substantially equivalent along the length of the anastomotic connector.

15. The anastomotic connector has a column pitch that varies along the length of the anastomotic connector.

16. The anastomotic of claim 1 wherein the outer diameter of the arterial portion is from 3-5 mm.

17. The anastomotic connector of claim 1 wherein the outer diameter of the open end of the venous portion is from about 5 to 7 mm.
